(19) United States
(12) Patent Application Publication
(10) Pub. No.: US 2012/0191066 A1
(43) Pub. Date: Jul. 26, 2012

(54) Prefilled Cannula Assembly

(75) Inventors: Michael Schabbach, Frankfurt am Main (DE); Daniel Wagner, Frankfurt am Main (DE); Gerrit Hauck, Frankfurt am Main (DE)

(73) Assignee: SANOFI-AVENTIS DEUTSCHLAND GMBH, Frankfurt am Main (DE)

(21) Appl. No.: 13/379,600

(22) PCT Filed: Jun. 24, 2010
(86) PCT No.: PCT/EP2010/058988

§ 371 Date: Apr. 15, 2012

Related U.S. Application Data
(60) Provisional application No. 61/220,442, filed on Jun. 25, 2009.

(30) Foreign Application Priority Data
Aug. 11, 2009 (EP) 09010324.3

(51) Int. Cl. A61M 5/31

(52) U.S. Cl. 604/506; 604/85

(57) ABSTRACT

A method and system for proving a cannula arrangement (4) for an injection device. The cannula arrangement comprises a limiting element (16) and a housing configured to attach to the injection device. The housing and the limiting element define a compressible reservoir (40). A second medication (26) is provided in the reservoir. A first cannula (12) comprises a piercing end (20) configured for fluid engagement with a medication provided in the injection device and a distal end (22) configured for fluid engagement with the reservoir. A second cannula (14) comprises a proximal end (48) for fluid engagement with the second medication and a piercing distal end (50) for the injection to a patient.

### FIELD OF THE PATENT APPLICATION

The present patent application generally describes an apparatus and/or method of delivering multiple medicaments (i.e., at least two medicaments). Preferably, such medicaments are stored or housed in a first container such as a cartridge or ampoule that is separate from a second container or reservoir. In one arrangement, these multiple medicaments are injected by way of a single injection. Preferably, one medicament is provided within a prefilled cannula assembly. This prefilled cannula assembly is attached to a dose injection device, preferably a multiple dose injection device. Such an injection device may be either a disposable or a reusable injection device, such as a pen type injection device comprising a cartridge or ampoule containing a medicament.

### BACKGROUND

There exists a general recognized need to inject two or more medications simultaneously. As just one example, certain disease states require treatments using one or more different medicaments. It can be beneficial to treat diabetics with both a long acting insulin and a glucagon-like peptide-1 (GLP-1), which is derived from the transcription product of the proglucagon gene, or a GLP-1 analog. GLP-1 is found in the body and is secreted by the intestinal L cell as a gut hormone. GLP-1 possesses several physiological properties that make it and its analogs interesting compounds for the treatment of diabetes mellitus.

Although our invention specifically mentions insulin, and GLP-1 or GLP-1 analogs as two possible drug combinations, other drugs or drug combinations, such as analgesics, hormones, beta agonists or corticosteroids, or a combination of any of the above-mentioned drugs could be used with our invention.

For the purposes of this invention the term “insulin” shall mean insulin, an insulin analog, an insulin derivative or a mixture thereof, including human insulin or a human insulin analogs or derivatives. Examples of insulin analogs are, without limitation, Gly(A21), Arg(B31), Arg(B32) human insulin; Lys(B3), Glu(B29) human insulin;

Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin or Des(B30) human insulin. Examples of insulin derivatives are, without limitation, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(w-carboxyheptadecanoyl)-des(B30) human insulin and B29-N-(w-carboxyheptadecanoyl) human insulin.

As used herein the term “GLP-1 analog” shall mean an analog of glucagon-like peptide-1 (GLP-1), including without limitation, exenatide (Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2), Exendin-3, Liraglutide, or AVE0010 (H-H is-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-Lys-Lys-Lys-ys-Lys-Lys-NH2).

Examples of beta agonists are, without limitation, salbutamol, levosalbutamol, terbutaline, pirbuterol, procaterol, metaproterenol, fenoterol, bitolterol mesylate, salmeterol, formoterol, bambuterol, clenbuterol, indacaterol.

Hormones are for example hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

Delivering at least two active medicaments or “agents” simultaneously can often create a number of challenges for the device provider as well as the user of the device. As just one example, the two active agents may interact with each other during the long-term, shelf life storage of the formulation. Therefore, it may be advantageous to store the active components separately and only combine these active components at the point of delivery. That is the case for formulations for injection, but is also true for formulations designated to other routes of administration, such as e.g. inhalation. However, from the standpoint of the user, combining the two agents should be straightforward and convenient so as to result in a reliable, accurate injection.

A further concern is that the quantities and/or proportions of each active agent making up the combination therapy may need to be varied for each user or at different stages of a user's therapy. For example, one or more active ingredients may require a titration period to gradually increase a patient up to a “maintenance” dose. A further example would be if one active agent requires a non-adjustable fixed dose while the other agent is varied in response to a patient's changing symptoms and/or physical conditions. This concern may mean that pre-mixed formulations of multiple active agents may not be suitable as these pre-mixed formulations would have a fixed ratio of the active components, which could not be varied by the healthcare professional or patient.

Accordingly, there exists a general need to provide devices and methods for the joint delivery of two or more medicaments. According to at least one embodiment, the present apparatus and method overcomes the above-mentioned concerns by providing a solution to the above-described problems by providing an improved cannula assembly and associated injection device having a cannula that has a reservoir containing an active medication. This cannula is part of a cannula assembly that is attached to an injection device, such as a pen injection device containing a cartridge. These and other advantages will become evident from the following more detailed description of the invention. The general problem to be solved by the present invention is to provide a needle assembly where an administration of at least two medicaments is facilitated.

According to an exemplary embodiment, a cannula arrangement comprises a limiting element and a housing configured to attach to an injection device containing a first medication. The housing and the limiting element define a compressible reservoir. Preferably, the limiting element is movable from a first position to a second position or from a first shape to a second shape and thus makes sure that the reservoir delimited by the limiting element has a volume that can be compressed. The limiting element may be a flexible member such as a membrane or a rigid member, such as a bung. A second medication is provided in this reservoir. A first cannula comprises a piercing end configured for fluid engagement with the first medication provided in the injection device and a distal end configured for fluid engagement with the reservoir. A second cannula comprises a proximal end for fluid engagement with the second medication and a piercing distal end for the injection to a patient. According to a preferred embodiment, said housing of said cannula arrangement is configured to be removably attached to said injection device. According to another preferred embodiment, said injection device comprises a pen type injection device. According to yet another preferred embodiment, said injection device comprises a resettable injection device. Furthermore, said first cannula may have a first Gauge size, and said second cannula may have a second Gauge size, and said first Gauge size of said first cannula may be generally equal to said second Gauge size of said second cannula.

A sealing element may be provided at the proximal end of the first cannula. The sealing element may be used to ensure proper sealing of the cannula arrangement for storage, separating the two medicaments once the cannula arrangement is attached to the drug delivery device. Additionally or alternatively the sealing element serves for ensuring that medicament in the reservoir is expelled only through the second needle, i.e. for closing the fluid path for the second medication when the compressible reservoir is compressed.

One embodiment of a sealing element is a compressible seal. Other embodiments could comprise a flexible seal, a deformable seal, a breakable seal, or a one-way valve.

Another benefit of the compressible seal is that it returns to its original shape and volume once the cannula arrangement is detached from the drug delivery device. Thereby it seals the first cannula piercing end preventing needle stick injury, e.g.

According to an alternative construction, the sealing element may be provided at the distal end of the drug delivery device. The sealing element may serve for ensuring that medicament in the reservoir is expelled only through the second needle of the attached cannula arrangement when the compressible reservoir is compressed.

According to another embodiment of said cannula arrangement, said housing and said limiting element define at least a first and a second reservoir. In this case, a second medication may be provided in said first reservoir, and a third medication may be provided in said second reservoir.

Preferably, said cannula arrangement comprises a pressing member for exerting a force on said limiting element so as to expel said second medicament through said second cannula member.

Furthermore an arrangement of said cannula arrangement and an injection device is described, wherein said injecting device comprises a pressing member for exerting a force on said limiting element so as to expel said second medicament through said second cannula member.

Furthermore, an injection system is described, the system comprising an injection device, e.g. such as a drug delivery device, containing a first medication in a reservoir, such as a container, cartridge, or ampoule, and a housing configured for attaching said cannula arrangement.

In one embodiment, the cannula arrangement comprises a flexible member and a housing, said flexible member and said housing defining at least one compressible reservoir. Preferably, a second medication is provided in said compressible reservoir. A first cannula is provided, the cannula comprising a piercing proximal portion configured for fluid engagement with said first medication provided in said injection device, and a distal end configured for fluid engagement with said compressible reservoir. In addition, a second cannula is provided, the second cannula comprising a proximal end for fluid engagement with said compressible reservoir and a piercing distal end.

In an alternative embodiment, the cannula arrangement comprises a housing and a movable bung, said movable bung and said housing defining at least one compressible reservoir. Preferably, a second medication is provided in said compressible reservoir. A first cannula is provided, the cannula comprising a piercing proximal portion configured for fluid engagement with said first medication provided in said injection device, and a distal end configured for fluid engagement with said compressible reservoir. In addition, a second cannula is provided, the second cannula comprising a proximal end for fluid engagement with said compressible reservoir and a piercing distal end.

Furthermore, an injection system is described, the system comprising an injection device, e.g. such as a drug delivery device, containing a first medication in a reservoir, such as container, cartridge, or ampoule, and a housing configured for attaching said cannula arrangement.

Furthermore, a method for operating a cannula arrangement is described, the method including the following steps:

(a) Attaching the housing as described above to the injection device.

(b) Operating a pressing member to expel said second medicament through said second cannula from the compressible reservoir.

(c) Establishing a fluid communication between the first cannula and the reservoir of the injection device. This may be done e. g. by piercing or compressing the sealing element.

(d) Operating the injection device to expel a previously selected quantity of the first medication through the first cannula, the compressed reservoir and the second cannula.

The quantity of the first medication may be a previously selected quantity, e.g. by a user. Alternatively the quantity may be a fixed quantify, e.g. in a fixed dose device.

If used for injecting medicament into a patient's body, a patient or a healthcare professional could introduce the second cannula to an intended injection site of the patient between steps a) and b).

Nevertheless and in particular, the method may be used for test purposes only, i.e. for testing the cannula arrangement without any medical treatment of a human being or an animal.

These as well as other advantages of various aspects of the present invention will become apparent to those of ordinary skill in the art by reading the following detailed description, with appropriate reference to the accompanying drawings.

The scope of the invention is defined by the content of the claims. The invention is not limited to specific embodiments but comprises any combination of elements of different embodiments. Moreover, the invention comprises any combination of claims and any combination of features disclosed by the claims.

### BRIEF DESCRIPTION OF THE DRAWINGS

Exemplary arrangements are described herein with reference to the drawings, in which:

FIG. 1 illustrates a first arrangement of a prefilled cannula assembly;

FIG. 2 illustrates a second arrangement of a prefilled cannula assembly;

FIG. 3a illustrates an initial step of an injection system for use with the prefilled cannula assembly illustrated in FIG. 1;

FIG. 3b illustrates a subsequent injection step of the injection system illustrated in FIG. 3a;

FIG. 3c illustrates a subsequent injection step of the injection system illustrated in FIG. 3b; and

FIG. 4 illustrates an injection device that may be used with the injection system illustrated in FIGS. 3a-c.

### DETAILED DESCRIPTION

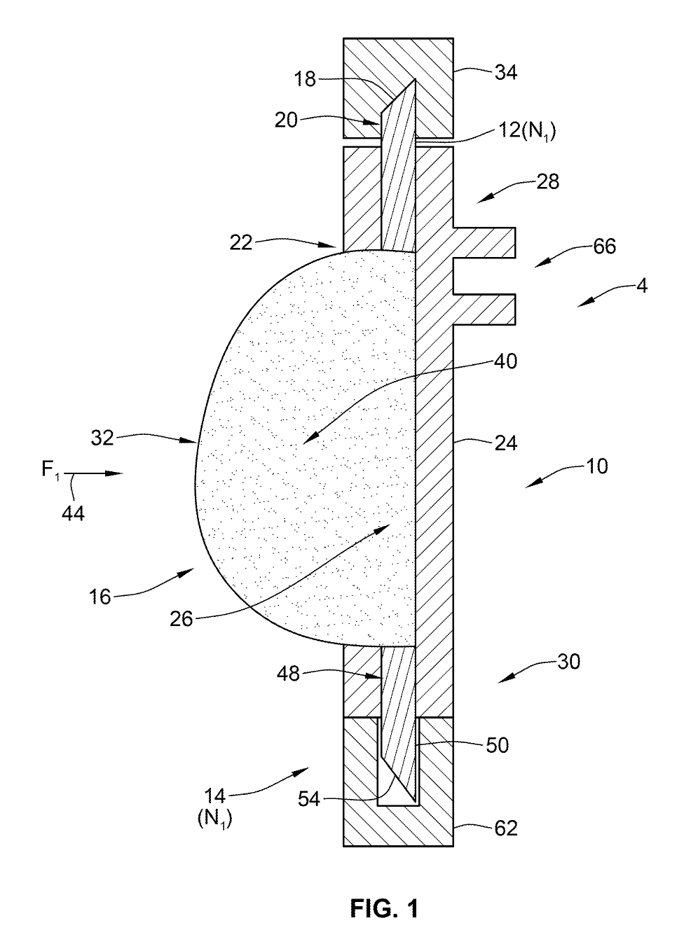

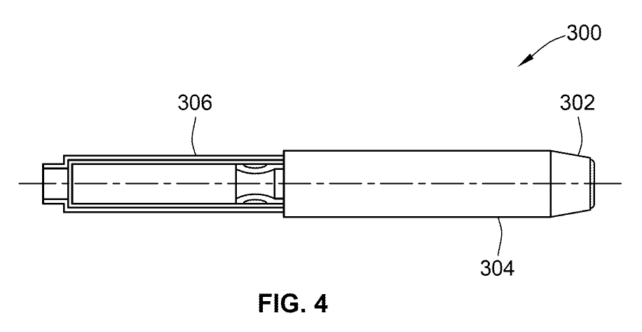

FIG. 1 illustrates a first arrangement of a prefilled cannula assembly 10. Such prefilled cannula assembly 10 is configured to be coupled to an injection device, such as the injection device illustrated in FIG. 4. Such prefilled cannula assembly 10 may be configured to be either releasably or non-releasably coupled to such an injection device.

In one arrangement, the injection device illustrated in FIG. 4 comprises a pen injection device. Such a typical pen type injection device comprises a cartridge or ampoule containing a first injectible liquid, such as insulin. As will be described in greater detail below, the injection device further comprises a dosing mechanism that allows a user of the device to set an injectible dose. The dose mechanism comprises a driving or injecting mechanism such that the injectible liquid contained in the cartridge and along with an injectible liquid contained in the attached cannula assembly may be injected during the same injection.

Returning to FIG. 1, the cannula assembly 10 comprises a cannula arrangement 4. In this illustrated arrangement, the cannula arrangement 4 comprises a first cannula 12 (N1) and a second cannula 14 (N2). However, as those of skill in the art will recognize, alternative cannula arrangements having three or more cannula may also be used with the general concept of the cannula arrangement described here.

In one exemplary arrangement, the first cannula 12 comprises a conventional cannula of 30 Gauge. The second cannula 14 may comprise a conventional cannula of similar size 30 Gauge or of alternative size (such as, e.g., 27 G, 28 G, 29 G, 30 G, 31 G or smaller). However, as those of ordinary skill in the art will recognize, other size cannula could also be used as well. As just one example, in an alternative arrangement, the first cannula 12 would comprise a cannula having a first gauge and the second cannula 14 would comprise a cannula having a second gauge size different than the first cannula.

The cannula assembly 10 further comprises a limiting element formed as a flexible member 16, e.g. such as a membrane, along with a housing element 24. In one preferred arrangement, the housing element 24 houses at least a portion of the first cannula 12 and at least a portion of the second cannula 14. The flexible member 16 is preferably disposed between the first and the second cannula 12, 14 and may be rigidly or removably affixed to the housing element 24.

As illustrated in FIG. 1, in this preferred arrangement, the flexible member 16 along with a portion of the housing 24 defines at least one medicament reservoir or cavity 40. Such a medicament reservoir or cavity 40 may be filled with a medication 26, such as a short acting insulin, a long acting insulin, or a GLP-1 or a GLP-1 analog. However, in an alternative arrangement, the flexible member 16 and housing 24 may define a plurality of medicament cavities, for example, at least two such medicament cavities. In such a multiple cavity or multiple reservoir arrangement, the first cavity may be filled with a one type of medication and the second cavity may be filled with a second type of medication.

Returning to FIG. 1, the first cannula 12 comprises at least a proximal end 20 and a distal end 22. The proximal end 20 of the first cannula 12 preferably comprises a sharpened and/or beveled end 18. The first cannula 12 is preferably mounted near a proximal portion 28 of the housing 24; however, alternative mounting arrangements may also be utilized. While mounted within the housing 24, the distal end 22 of the first cannula 12 can remain in fluid engagement with the medicament cavity 40 defined by the flexible member 16. Alternatively, the first cannula 12 is not in permanent contact with the medication upon storage, but will be in fluid engagement to the medication reservoir 40 during administration using a suitable activation mechanism, e.g. comprising a metal spring, a plastic spring element, gas pressure or the like.

In one preferred arrangement, the flexible member 16 is made of a suitable plastic material adequate for permanent contact with a parenteral drug product. Examples for such materials are polypropylene (PP), polyethylene (PE), polyurethane (PU), polyethylene terephtalate (PET), or polystyrene (PS). In a preferred arrangement, the medicament cavity 40 contains a quantity of another medicament 26. Preferably, a quantity of this second medicament 26 comprises a fixed dose of medication.

A compressible seal 34 is provided at the proximal end 20 of the first cannula 12. Preferably, this compressible seal 34 is provided along with the cannula assembly 10 when the user takes this assembly out of its shipping or storage packaging. In one preferred arrangement, this compressible seal 34 may be compressible along an outer surface of the first cannula 12. As will be explained in greater detail below, the medicament cavity 40 has a variable volume such that, as an external pressing member exerts a force F₁ 44 against an external wall 32 of the flexible member 16, the second medicament 26 contained within this cavity is driven out of the flexible member 16. Because the seal 34 is provided at the proximal end 20 of the first cannula, as the flexible member 16 is compressed under the pressure of this force F₁ 44, the medication contained within the flexible member 16 is pushed out of the second cannula portion 14 and then preferably into an injection site of a patient.

At the distal portion 30 of the housing 24, a second cannula 14 is provided. Similar to the first cannula 12, this second cannula 14 comprises a proximal end 48 and a distal end 50. While mounted within the distal portion 30 of the housing 24, the proximal end 48 of the second cannula 14 either remains in fluid engagement with the medicament cavity 40 or comes into contact with the fluid cavity 40 during administration by the aid of an activation mechanism. The distal end 50 is provided with a beveled and piercing end 54 so as to provide a piercing end for an injection site of a patient.

In addition, the second cannula 14 is provided with a removable cannula cap 62. This cannula cap 62 serves to protect the distal end 50 of the second cannula portion 14 during shipment or packaging. This removable cap 62 also can be used to prevent an inadvertent cannula stick. Before the cannula assembly 10 may be used for injection of the first and the second medicaments, a user must remove the cannula cap 62 before injection.

The housing 24 is made of one or more appropriate plastic materials suitable for medical devices. Examples for such materials are polypropylene (PP), polyethylene (PE), polyurethane (PU), polyethylene terephtalate (PET), or polystyrene (PS). Alternative materials could also be used. The housing 24 further includes a coupling mechanism 66. Preferably, this coupling mechanism 66 provides a user or a patient with a means to releasably connect the cannula assembly 10 to the injection device, such as the pen injection device illustrated in FIG. 4. Such coupling mechanism 66 could comprise a snap lock, or other similar type of releasably coupling.

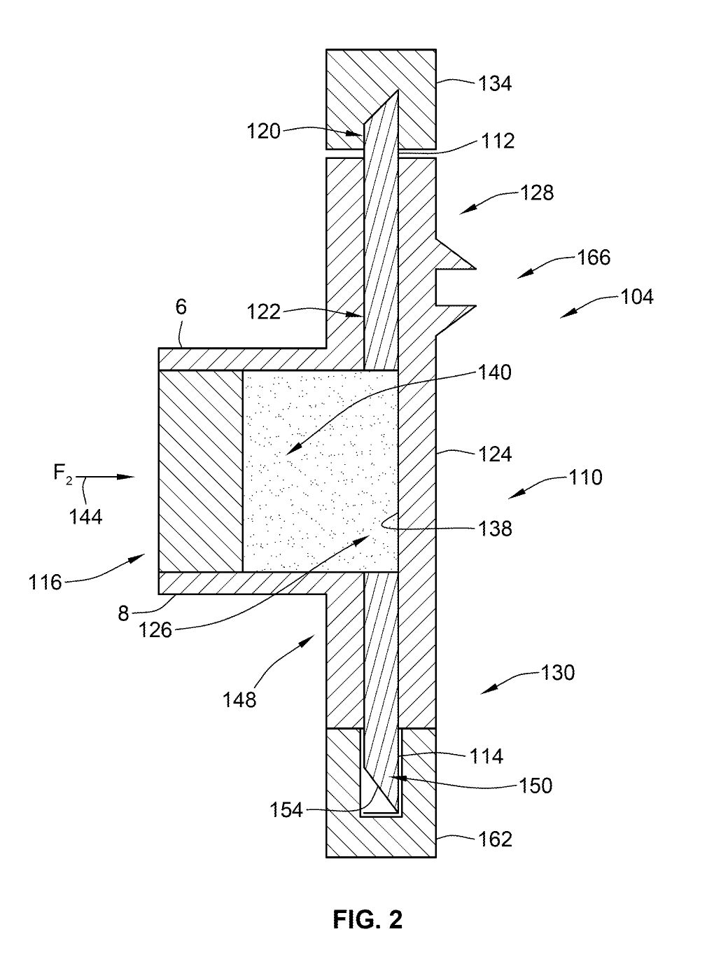

FIG. 2 illustrates an alternative arrangement of a prefilled cannula assembly 110. Such prefilled cannula assembly 110 is configured to be coupled to an injection device, such as an injection device illustrated in FIG. 4. Such prefilled cannula assembly 110 may be configured to be either releasably or non-releasably coupled to such an injection device.

As described above, in one arrangement, the injection device illustrated in FIG. 4 comprises a pen injection device. Such a typical pen type injection device comprises a cartridge or ampoule containing a first injectible liquid, such as insulin. As will be described in greater detail below, the injection device further comprises a dosing mechanism that allows a user of the device to set an injectible dose.

Returning to FIG. 2, the cannula assembly 110 comprises a cannula arrangement 104. In this illustrated arrangement, the cannula arrangement 104 comprises a first cannula 112 (N1) and a second cannula 114 (N2). However, as those of skill in the art will recognize, alternative cannula arrangements having at least three or more cannula sections may also be used with the general concept of the cannula arrangement described here.

In one arrangement, the first cannula 112 comprises a conventional cannula of 30 Gauge. The second cannula section 114 may comprise a conventional cannula of similar size 30 Gauge or of alternative size (such as, e.g., 27 G, 28 G, 29 G, 30 G, 31 G or smaller). However, as those of ordinary skill in the art will recognize, other size cannula could also be used as well. As just one example, in an alternative arrangement, the first cannula 112 would comprise a cannula having a first gauge and the second cannula 114 would comprise a cannula having a second gauge size different than the first cannula.

The cannula assembly 110 further comprises a bung member 116 along with a housing element 124. In one preferred arrangement, the housing element 124 houses at least a portion of the first cannula 112 and at least a portion of the second cannula 114. The bung member 116 is preferably disposed partially between the first and the second cannula 112, 114. This bung member 116 is slidable between a proximal housing member 6 and a distal housing member 8. For example, if a force F2 144 acts on the bung member 116, the bung member will move from its starting position (as illustrated in FIG. 2) to its end position where the bung member will eventually reside adjacent an end wall 138 of the housing 124.

As illustrated in FIG. 2, in this preferred arrangement, the bung member 116 along with the housing 124 define at least one medicament reservoir or cavity 140. Such a medicament reservoir or cavity may be filled with a medication 126, such as a short acting insulin or a long acting insulin or another active ingredient such as an GLP-1 or a GLP-1 analog. However, in an alternative arrangement, the bung member 116 may define a plurality of medicament cavities, for example, at least two such medicament cavities. In such a medicament a multiple cavity arrangement, the first cavity may be filled with a one type of medication and the second cavity may be filled with a second type of medication.

Returning to FIG. 2, the first cannula 112 comprises at least a proximal end 120 and a distal end 122. The proximal end 120 of the first cannula 112 preferably comprises a sharpened and/or beveled end. The first cannula 112 is preferably mounted near a proximal portion 128 of the housing 124; however, alternative mounting arrangements may also be utilized. While mounted within the housing 124, the distal end 122 of the first cannula 112 can permanently remain in fluid engagement with the medicament cavity 140 defined by the bung member 116. Alternatively, the first cannula 112 is not in permanent contact with the medication upon storage, but will be connected to the medication reservoir 140 during administration using a suitable activation mechanism, e.g comprising a metal spring, a plastic spring element, gas pressure or the like.

In one preferred arrangement, the bung member 116 is made of a suitable flexible plastic material made by an extrusion process. Different polymers can be used for the bung member, e.g. polypropylene (PP), polyethylene (PE), polyurethane (PU), polyethylene terephtalate (PET), or polystyrene (PS). Co-extrusion of different polymers can be applied to optimize the properties of the reservoir, e.g. with respect to vapor or oxygen permeability. In a preferred arrangement, the medicament cavity 140 defined by the bung member 116 and the housing 124 contains a quantity of another medicament 126. Preferably, a quantity of this second medicament 126 comprises a fixed dose of medication.

A compressible seal 134 is provided at the proximal end 120 of the first cannula 112. Preferably, this compressible seal 134 is provided along with the cannula assembly 110 when the user takes this assembly out of its shipping or storage packaging. In one preferred arrangement, this compressible seal 134 may be compressible along an outer surface of the first cannula member 112. As will be explained in greater detail below, the medicament cavity 140 has a variable volume such that, as an external pressing member exerts a force F 144 against the bung member 116, the second medicament 126 contained within this cavity is driven from the cavity. Because the seal 134 is provided at the proximal end 120 of the first cannula 112, as the bung member 116 moves towards the wall 138 of housing 124 under the force of F 144, the medication contained within the bung member 116 is expelled from the cavity and into the second cannula 114 and, preferably into an injection site of a patient.

At the distal portion 130 of the housing 124, a second cannula 114 is provided. Similar to the first cannula 112, this second cannula 114 comprises a proximal end 148 and a distal end 150. While mounted within the distal portion 130 of the housing 124, the proximal end 148 of the second cannula 114 can remain in fluid engagement with the medicament cavity 140 defined by the bung member 116 and housing 124. Alternatively, the second cannula 114 is not in permanent contact with the medication upon storage, but will be connected to the medication reservoir 140 during administration using a suitable activation mechanism, e. g comprising a metal spring, a plastic spring element, gas pressure or the like. The distal end 150 is provided with a beveled and piercing end 154 so as to provide a piercing end for an injection site of a patient. In addition, the second cannula 114 is provided with a removable cannula cap 162.

The housing 124 may be made from one or more appropriate plastic materials suitable for medical devices. Examples for such materials are polypropylene (PP), polyethylene (PE), polyurethane (PU), polyethylene terephtalate (PET), or polystyrene (PS). Alternative materials could also be used. The housing 124 further includes a coupling mechanism 166. Preferably, this coupling mechanism 164 provides a user or a patient with a means to releasably connect the cannula assembly 110 to the injection device, such as the pen injection device illustrated in FIG. 4. Such coupling mechanism 166 could comprise a snap lock, or other similar type of releasably coupling. Alternatively, such coupling mechanism could comprise a means to permanently connect the cannula assembly 110 to the injection device. Such a permanent connection would provide a certain guarantee that the cannula assembly along with the injection device could only be used for a single injection.

For performing an injection, in addition to a first medicine, an additional dose of another, a second medicine may be administered. Both the first and the second medicines will be injected parenterally (e.g., subcutaneously). This can be achieved with the aid of an injection device so that only one injection is required. Depending on the specific type of therapy, the medicine is to be administered variably selectable depending on an individual patient's need. The second medicine, on the other hand, is to be administered in addition as a fixed dose. The first and second medicines are to be injectable by way of the same cannula assembly by way of a single injection step.

In one arrangement, the injection device allows for a variable dose selection of the first medicament that is to constructed similarly to already existing, established injection devices. Examples of such existing injection devices may comprise variably selectable auto injectors (e.g. auto pens) as well as variably selectable injection aids that are operated manually (e.g., insulin pens).

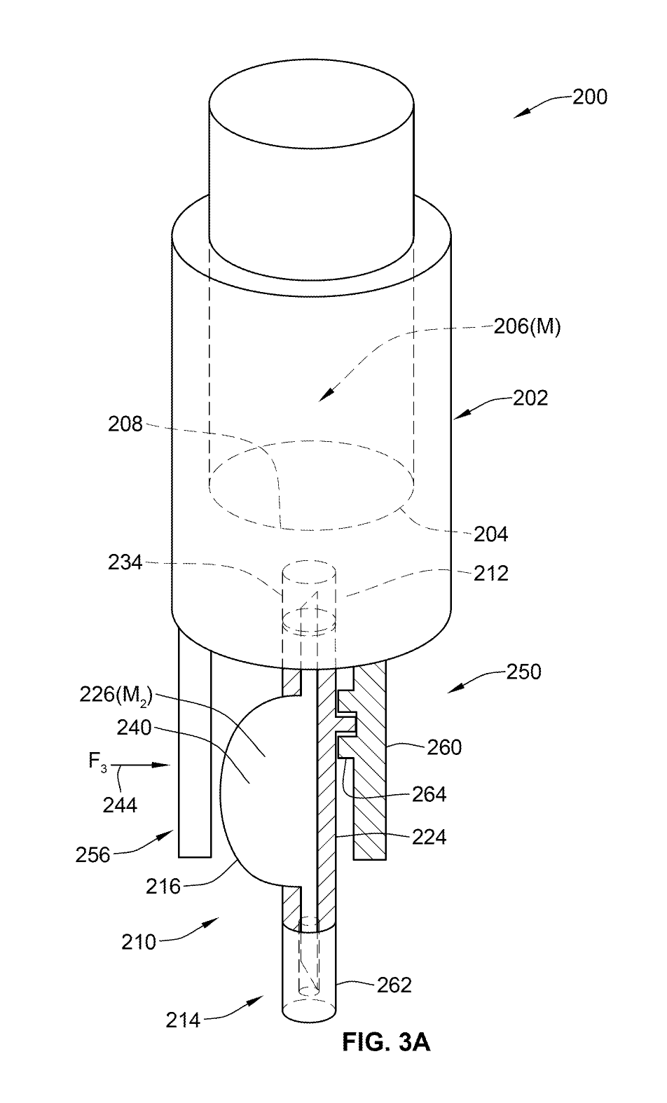

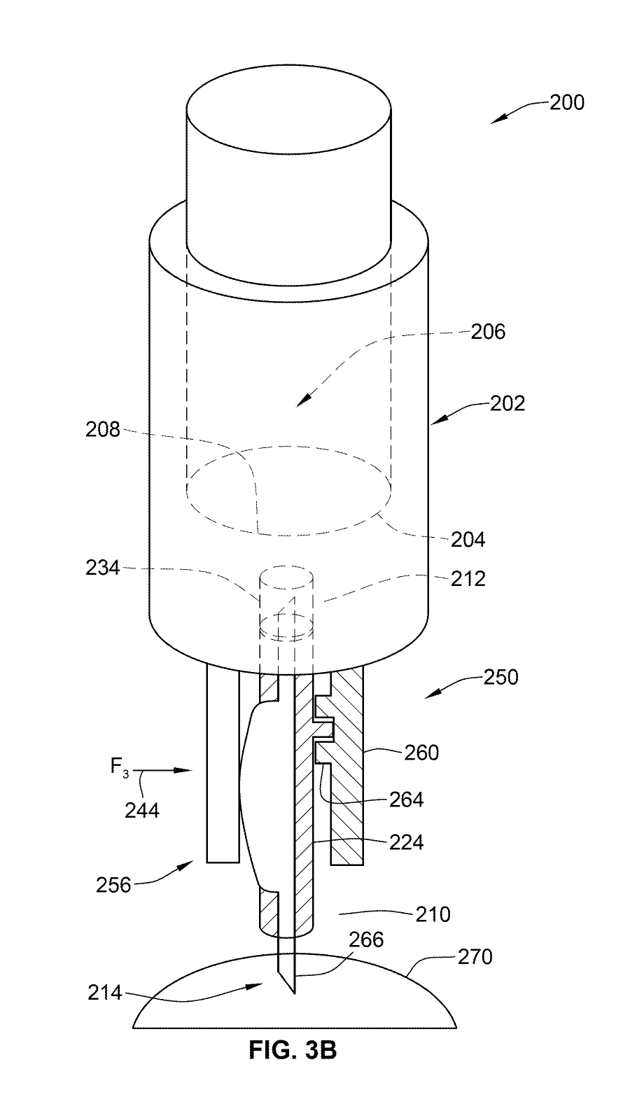

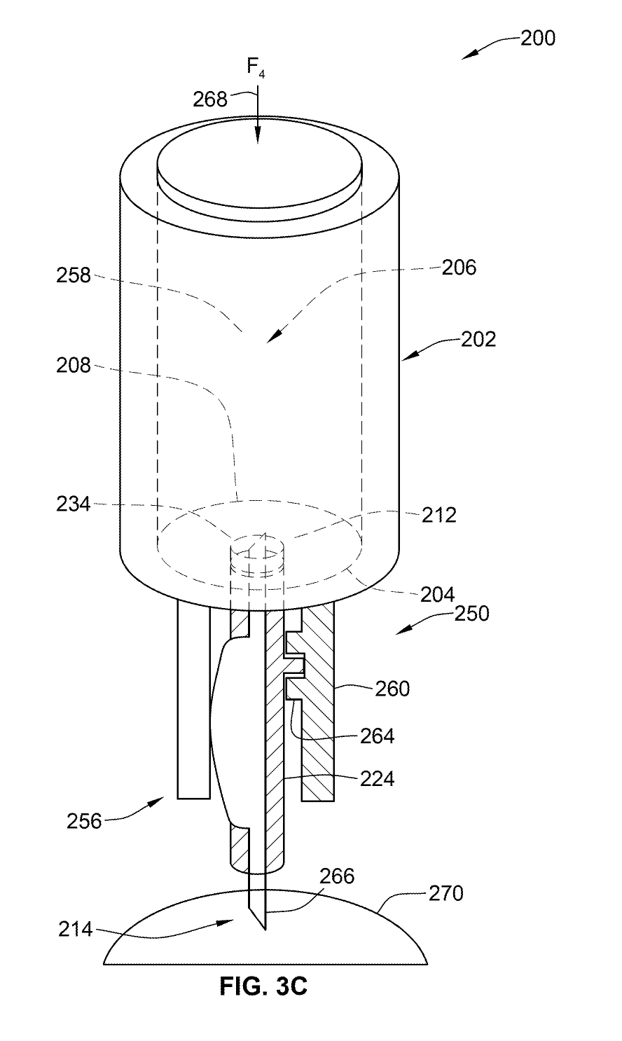

FIG. 3a illustrates an injection system 200 for use with the prefilled cannula assemblies illustrated in FIGS. 1 and 2. The process of injecting the first medication contained in the cartridge of the injecting device and the second medication contained within the cannula assembly may occur in the following manner.

Initially, a prefilled cannula assembly is received that contains the cannula assembly. Such a cannula assembly could comprise the prefilled cannula assemblies 10, 110 illustrated in FIGS. 1 and 2, respectively. In this arrangement, the cannula 210 comprises a prefilled cannula assembly, similar to the assembly 10 illustrated in FIG. 1. As such, the prefilled cannula assembly 210 comprises a first cannula member 212, a second cannula member 214, flexible member 216 along with a housing element 224. In this arrangement, the housing element 224 houses at least a portion of the first cannula member 212 and at least a portion of the second cannula member 214. The flexible member 216 is preferably disposed between the first and the second cannula sections 212, 214 and may be rigidly or removably affixed to the housing element 224.

In a first initial step, the cannula assembly 210 is removed from its packaging and is mounted onto or into the injection device 202. Such injection device may comprise a pen type injection as illustrated in FIG. 4. During this mounting step, the coupling mechanism 264 of the cannula assembly 210 (similar to the coupling mechanisms 66, 166 of cannula assemblies 10, 110) is attached to the injection device 202. Initially, the cannula assembly 210 is packed in a sterile enclosure and can easily be taken out by the user, where such a sterile enclosure may comprise a blister pack, a bag, or other similar type enclosure.

The prefilled cannula assembly 210 is preferably filled with a medication 226, such as an insulin, a GLP-1, or a GLP-1 analog. The proximal end of the first cannula 212 is aligned with a septum 208 located at a distal end of a conventional cartridge or ampoule 204 housed within the injection device 202. Such a conventional cartridge or ampoule 204 containing the first medicine can be, for example, a 3 ml insulin cartridge which is conventionally used in insulin pens. Those of ordinary skill in the art will recognize that cartridge or ampoules of other formats or conventions may also be used.

In one arrangement, the coupling mechanism 264 of the cannula assembly 210 allows the assembly to be snapped onto or into a receiving mechanism 260 of the injection device. Such a receiving mechanism may be internal or external to the injection device 202. The user then removes the removable cover 262 from the second cannula 214 of the cannula assembly 210. Utilizing the injection device 202, the user can now select a required quantity of the first medicine 206 contained within the cartridge 204.

As just one example, and referring to the injection device 300 illustrated in FIG. 4, the dose may be selected in the following manner. The injection device 300 could be a reusable or disposable device. By disposable device it is meant an injection device that is obtained from the manufacturer preloaded with a medicament and cannot be reloaded with new medicament after the initial medicament is exhausted. The device may be a fixed dose or a settable dose, but in either case it is a multi-dose device.

In FIG. 4, the conventional device 300 comprises a cartridge housing 306, a dose dialing module 304, and a dose adjustment knob 302. A first end of the cartridge housing 306 and the dose dialing module 304 are secured together by retaining features. Such typical injection device 300 contains a cartridge or other reservoir of medication. This cartridge is typically cylindrical in shape and is usually manufactured in glass or an essentially translucent plastic material. The cartridge is sealed at one end with a rubber bung and at the other end by a rubber septum. The injection pen is designed to deliver multiple injections. It therefore has features, for example a screw thread, which are used to attach an injection cannula assembly, such as the cannula assembly illustrated in FIG. 1. As discussed with reference to FIGS. 1 and 2, the disclosed prefilled cannula assembly is designed to pierce the cartridge septum and provide fluid communication between the contents of the cartridge and the subcutaneous region of the patient. The medicament contained with the cartridge housing 306 is expelled by a mechanism in the injection device that causes the cartridge bung to advance. The delivery mechanism is typically powered by a manual action of a user; however, the injection mechanism may also be powered by other means such as a spring, compressed gas or electrical energy.

In a preferred arrangement, the cannula assembly 210 of FIG. 3a can be designed to work with only a particularly designed injection device that is coded to work with only a single type cannula assembly. In an alternative arrangement, the cannula assembly 210 may be designed to work with only a particularly designed injection device that is coded to work with only a limited number of cannula assemblies. This could be achieved by including specific connecting or coupling features on the injection device that engage matching, complementary, or coding features on the cannula assembly 210. For example, specific coupling, connecting, or coding features may be provided on the distal end 250 of the injection device 200 that engage matching or complementary features on a specific type of cannula assembly for use with only one type of injection device. As just one example, a specific type of cannula assembly 210 containing a fast acting insulin as an active ingredient may only be allowed to be connected to only a specific type of injection device containing a long acting insulin. However, those of skill in the relevant art will recognize alternative mechanical arrangements and active ingredient arrangements are also possible.

One reason for restricting the use of the injection system to a particular cannula assembly is to ensure dose accuracy of the medicament delivered from the injection device. Another reason for restricting the use of the injection system to a particular cannula assembly is to ensure that only certain active medicaments can be delivered along with only certain other active medicaments stored within the injection device.

Once the user has set the dose to be administered of the first medication 206 contained within the cartridge 204, the second cannula 214 is introduced into an intended injection site 266 of a patient 270. This is illustrated in FIG. 3b. Next, the injection process is activated by pressing on a corresponding trigger of the injection device 202. The injection device 202 first exerts a force F₃ 244 against the flexible member 216. This force F₃ 244 acts to expel the second medicament 226 out of the reservoir or cavity 240 through the second cannula 214 into the injection site of the patient.

In one arrangement, a pressure pad 256 could be used as pressing member to exert a force on the flexible member 216. For example, such a pressure pad 256 could be provided on the injection device 202. The pressure pad 256 may be driven by the injection device 202, in this preferred arrangement. Alternatively, such a pressure pad 256 could be a separate component part or an attachment to the injection device 202. Patient safety can be enhanced by allowing the user to check if the cavity 240 is empty, incompletely filled, or contaminated with particulate matter. For this purpose the reservoir 240 needs to be manufactured from an essentially translucent material allowing a visible inspection.

In yet an alternative arrangement, the pressure pad 256 could be an integrated component of the cannula assembly 210. In this case, the pressure pad could be advantageously locked and/or loaded with a pre-loaded biasing means, such as a spring. The interlocking can therefore be released by the patient by pressing out the cannula assembly from its packaging. In one arrangement, the biasing means could be a metal spring (e.g., a leaf spring, cylindrical helical spring, conical spring), a plastic spring element, or a gas pressure spring.

Returning to the injection system illustrated in FIG. 3b, in the subsequent injection step, a slight pressure is built up on the cartridge 204. This slight pressure prevents discharge of the medicament 206 from the cartridge 204 into the medicament reservoir 240 and the second medication 226.

In a subsequent injection step illustrated in FIG. 3c, the injection device 202 alters the location of the cartridge 204 and utilizes its dosing mechanism to move the cartridge 204 in a distal direction. Preferably, the dosing mechanism moves the cartridge 204 in a distal direction so that the cartridge membrane 208 resides over the rubber seal 234 surrounding the first cannula 212. In this manner, the first cannula 212 pierces the membrane 208 so that the first cannula 212 will now be in fluid engagement with the medicament 206 provided in an inner cavity 258 of the cartridge 204.

The rubber seal 234 acts as a sealing member that surrounds the first cannula 212 is pushed in the distal direction and will subsequently reside in a compressed state, surrounding the first cannula 212 but allowing the cannula 212 to pierce the cartridge membrane 208. As a result of the pressure that is already built up on the first medication 206 contained within the cartridge 204, the second medication 226 contained within the cannula reservoir cannot flow back into the cartridge of the injection device and must therefore be expelled through the second cannula 214.

Next, the dosing mechanism (preferably by way of a plunger rod) of the injection device 202 begins to act on the bung contained within the cartridge 204 by way of force F₄ 268. In this manner, the dosing mechanism of the injection device 202 injects the previously selected quantity of the first medication 206 from the cartridge 204 first through the first cannula 212, through the compressed reservoir 216, and then through the second cannula 214. This medicament is then injected by way of the second cannula portion 214 into an injection site 266 of a patient 270.

Preferably, this injection process takes place manually. However, in an alternative injection system arrangement, the injection process can take place automatically. After injection, the cartridge 204 is pulled back in a proximal direction so that the cartridge will eventually reside in its initial position, as illustrated in FIG. 3A and 3B. The rubber seal 234, now being not compressed, will return to its original shape covering the first cannula 212. Thereby it seals and covers the cannula's proximal end providing for a liquid tight closure of the proximal end of the cannula arrangement and/or protection against needle stick injuries. Then, the user can remove the cannula assembly 210 from of the injection site 266 and remove the cannula assembly 210 from the injection device 202. The cannula assembly 210 can then be disposed.

One advantage of the prefilled cannula assembly described here is that the administration of the reservoir medication does not require additional complicated handling steps compared with the administration of a single dose of the first medicine. This is true in that the cannula assembly with the two cannulas can be handled principally identical to a standard pen type cannula assembly for use with a pen type injection device. This ease of use results in increased patient acceptance and can also increase operating safety of both the injection device as well as the prefilled cannula assembly.

The arrangement discussed above utilizes a connecting mechanism 264 that operates with a receiving mechanism 260 of the injection device. Alternative connector arrangements may also be used. In such arrangements, this connector arrangement can be any design known to the art, preferably one that is releasable by a user. For example, such a releasable connector could comprise a single or multiple start thread, a bayonet lock, a luer lock, ramps and detents, snap locks, snap fits or other connector that has a male or female part that connects to the corresponding female or male part on the medicament housing.

With certain presently known injection devices, such as the injection device illustrated in FIG. 4, ensuring an exact dose of two active ingredients presents certain challenges as described above. The disclosed cannula assembly provides a fixed dose available in a reservoir or inner space of the cannula assembly that contains an active ingredient in a cannula that can be connected onto an injection device, such as a pen injection device. This pen device may then be used to set a variable dose of a medication contained within the pen device, for example a medication contained within a cartridge or ampoule. The device can then be used to administer this variably set dose along with the active ingredient stored within the cannula assembly reservoir.

There are a number of advantages to the presently claimed cannula assembly. For example, one advantage is that exact dosing of low-dose active ingredients may be achieved. Dosing is ensured by the single dose and is not dependent on tolerances of the dosing system (device) or variability of multi-dose packaging, e.g. a cartridge/stopper system.

In addition, the presently disclosed cannula assemblies allow for the combination of a fixed dose with a variable dose. In other words, a fixed dose can be combined with a large range of variable doses. An advantage of the prefilled cannula assembly is that by featuring a flexible container for the fixed dose medication, losses of the variable medication are minimized and the minimum volume of the variable medication required to flush the entire volume of the fixed medication from its reservoir is significantly lowered due to the reduced dead volume after the fixed medication has been expelled from its reservoir. Other technical solutions, such as, for example, devices with several cartridges for fixed and variable doses are expensive to develop and to manufacture as well as complicated to handle.

The disclosed cannula assembly results in a simple and safe to use application. In other words, by simply mounting of the cannula system with an active ingredient reservoir onto an injection device, operation is scarcely different from known device systems and is conceivably easy. One potential result is a higher safety of use.

The proposed injection system can be designed to work with currently marketed pen-type injection devices or may be designed to work only with one particular design of injection device. The latter could be achieved by including specific features on the injection device that engage matching or complementary features on the prefilled cannula assembly device.

Exemplary embodiments of the present invention have been described. Those skilled in the art will understand, however, that changes and modifications may be made to these embodiments without departing from the true scope and spirit of the present invention, which is defined by the claims.

### REFERENCE NUMERALS

4 cannula arrangement

6 proximal housing member

8 distal housing member

10 prefilled cannula assembly

12 first cannula

14 second cannula

16 flexible member

18 sharpened and/or beveled end

22 distal end

24 housing element

26 second medication

28 proximal portion of the first cannula

30 distal portion of the first cannula

34 compressible seal

40 medicament reservoir/cavity

44 force F1

48 proximal end of the second cannula

50 distal end of the second cannula

54 piercing end

62 removable cannula cap

66 coupling mechanism

104 cannula arrangement

110 cannula assembly

112 first cannula

114 second cannula

116 bung member

120 proximal end of the first cannula

122 distal end of the first cannula

124 housing element

126 second medication

128 proximal portion of the housing

134 compressible seal

138 end wall

140 medicament reservoir or cavity

144 force F2

148 proximal end of the second cannula

150 distal end of the second cannula

154 piercing end

162 removable cannula cap

164 coupling mechanism

166 coupling mechanism

200 injection system

202 injection device

204 conventional cartridge or ampoule

206 first medicine

208 septum

210 cannula assembly

212 first cannula member

214 second cannula member

216 flexible member

220 proximal end of the first cannula

224 housing element

226 second medication

234 rubber seal

240 cavity

244 force F3

250 distal end

256 pressure pad

258 inner cavity

260 receiving mechanism

262 removable cover

264 coupling mechanism

266 injection site

268 force F4

270 patient

300 injection device

302 dose adjustment knob

304 dose dialing module

306 cartridge housing

1. A cannula arrangement for an injection device containing a first medication comprising,
a limiting element;
a housing configured to attach to said injection device, said housing and said limiting element defining a compressible reservoir;
a second medication provided in said reservoir;
a first cannula comprising
a piercing proximal end configured for fluid engagement with the first medication provided in said injection device, and
a distal end configured for fluid engagement with said second medication provided in said reservoir; and
a second cannula comprising
a proximal end for fluid engagement with said second medication provided in said reservoir, and
a piercing distal end.

2. The arrangement of claim 1 wherein a sealing element is provided at the proximal end of the first cannula for closing the fluid path for the second medication during compression of the compressible reservoir.

3. The arrangement of claim 2 wherein the sealing element comprises one of a compressible seal, a flexible seal, a deformable seal, a breakable seal, a one-way valve.

4. The arrangement of claim 1 wherein said compressible reservoir comprises an insulin and/or an GLP-1 or an GLP-1 analog.

5. The arrangement of claim 1 wherein said compressible reservoir comprises a fixed dose of said second medication.

6. The arrangement of claim 1, wherein said first cannula has a first gauge size, and said second cannula has a second gauge size, wherein said first gauge size of said first cannula is generally equal to said second gauge size of said second cannula.

7. The arrangement of claim 1 wherein said housing and said limiting element define at least a first and a second reservoir.

8. The arrangement of claim 7 wherein a second medication is provided in said first reservoir, and a third medication is provided in said second reservoir.

9. The arrangement of claim 1 wherein said limiting element comprises a flexible member.

10. The arrangement of claim 1 wherein said limiting element comprises a movable bung.

11. The arrangement of claim 1 wherein an injection device is provided, the injection device containing a first medication in a reservoir, and wherein the housing is attached to said injection device such that the injection device and the cannula arrangement form an injection system.

12. The arrangement of claim 1 wherein said cannula arrangement comprises a pressing member for exerting a force on said limiting element so as to expel said second medicament through said second cannula member from the compressible reservoir.

13. The arrangement of claim 11 wherein said injecting device comprises a pressing member for exerting a force on said limiting element so as to expel said second medicament through said second cannula member from the compressible reservoir.

14. The arrangement of claim 11 wherein said injection device is configured to allow a user to set a variable dose of said first medication.

15. A method for operating the arrangement of claim 11, including the following steps:
(a) Attaching the housing to the injection device;
(b) Operating the pressing member to expel said second medicament through said second cannula from the compressible reservoir;
(c) Establishing a fluid communication between the first cannula and the reservoir of the injection device;
(d) Operating the injection device to expel a quantity of the first medication through the first cannula, the compressed reservoir and the second cannula.
